(19) United States
(12) Patent Application Publication
(10) Pub. No.: US 2010/0211131 A1
(43) Pub. Date: Aug. 19, 2010

(54) INTRAVASCULAR SYSTEM AND METHOD FOR BLOOD PRESSURE CONTROL

(76) Inventors: Michael S. Williams, Santa Rosa, CA (US); Richard A. Glenn, Santa Rosa, CA (US); Daniel W. Fifer, Windsor, CA (US)

Correspondence Address: SYNECOR LLC, P.O. BOX 5325, LARKSPUR, CA, 94977, US

(21) Appl. No.: 12/419,717

(22) Filed: Apr. 7, 2009

Related U.S. Application Data
(60) Provisional application No. 61/043,070, filed on Apr. 7, 2008.
(60) Provisional application No. 61/043,350, filed on Apr. 8, 2008.

(51) Int. Cl. A61N 1/05

(52) U.S. Cl. 607/44

(57) ABSTRACT

An intravascular lead is used to deliver energy for stimulating nervous system targets using energy delivery elements (e.g. electrodes) that are in direct contact with the nervous system targets. The lead may be positioned within the internal jugular vein and the nervous system targets may include the carotid artery/carotid sinus bulb and/or associated baroreceptor afferents, and/or surrounding nervous system targets in the region of the internal jugular vein, such as the carotid sinus nerve and/or associated nerve branches and/or the vagus nerve and/or associated nerve branches. Stimulation energy travels along a conductive bridge that extends from the intravascular lead to the nervous system target, or is relayed from the intravascular lead to another device disposed within or surrounding the target structure.

### RELATED APPLICATIONS

This application claims the benefit of U.S. Provisional Application No. 61/043,070, filed Apr. 7, 2008, and U.S. Provisional Application No. 61/043,350, filed Apr. 7, 2008, each of which is incorporated herein by reference.

### FIELD OF THE INVENTION

The present invention generally relates to implantable devices and systems, and associated methods for delivering therapy to nervous system targets using components implanted within the vasculature.

### BACKGROUND OF THE INVENTION

Heart failure (HF) is a condition characterized by reduced cardiac output that triggers neurohormonal activation. This compensatory mechanism functions acutely to increase cardiac output and restore left ventricular (LV) functional capacity such that patients remain asymptomatic. Over time, however, sustained activation of these neurohormonal systems triggers pathologic LV remodeling and end-organ damage that ultimately drives the progression of HF.

In many people, persistent hypertension is the predominant contributing factor for development of HF. Management of hypertension can slow or prevent the natural evolution of HF.

The human body maintains blood pressure through the use of a central control mechanism located in the brain with numerous peripheral blood pressure sensing components. These components are generally made of specialized cells embedded in the walls of blood vessels that create action potentials at an increased rate as the cell is stretched. These groups of cells are generally referred to as baroreceptors. The action potentials are propagated back to the central control center via neural pathways along afferent nerves. While there are many baroreceptor components located throughout the body, there are several that are particularly important. Possibly the most important baroreceptor region is located near the bifurcation of the common carotid artery into the internal and external carotid. In this area there is a small enlargement of the vessel tissues, referred to the carotid bulb or carotid sinus, the carotid baroreceptors are generally found throughout this area. The carotid baroreceptors and related neural pathways form the primary pressure sensing component that provides signals to the brain for regulating cranial and systemic blood pressure.

The baroreceptors in the aorta are the second best understood baroreceptors and are also a powerful localized blood pressure sensing component. The aortic baroreceptors are also responsible for providing signals to the brain for regulating systemic/peripheral blood pressure.

Applicant's prior Application Publication No. U.S. 2007/0255379, discloses an intravascular neurostimulation device (such as a pulse generator) and associated methods for using the neurostimulation device to stimulate nervous system targets. As discussed in that application, targeting stimulation to baroreceptor afferents in HF patients can lead to decreases in sympathetic tone, peripheral vascular resistance, and afterload. Such stimulation can be used to control blood pressure as a treatment for hypertension or HF. Stimulation of the vagus nerve (e.g. vagal efferents) is known to cause a reduction in heart rate and an increase in parasympathetic tone.

The present disclosure describes an implementation of Applicants' previously-disclosed intravascular systems and methods for stimulating nervous system targets such as baroreceptor afferents such as those associated with the carotid sinus, the carotid artery, the carotid sinus nerve or its branches, baroreceptors, and/or for otherwise activating a baroreceptor response and/or for stimulating the vagus nerve and/or its branches. Systems and methods of the type disclosed may be used for controlling heart rate and/or regulating blood pressure for treatment of hypertension, heart failure or other conditions.

The internal jugular vein, vagus nerve, and common carotid artery (which includes the carotid sinus) are located within the carotid sheath, a fascial compartment within the neck. The carotid sheath provides relatively fixed geometric relationships between these structures while also giving some degree of insulation from surrounding tissue.

Applicant's co-pending application Ser. No. 12/413,495, filed Mar. 27, 2009 and entitled SYSTEM AND METHOD FOR TRANS VASCULARLY STIMULATING CONTENTS OF THE CAROTID SHEATH discloses a method for transvascularly stimulating contents of the carotid sheath. The disclosed method includes advancing an energy delivery element, which may be an electrode, into an internal jugular vein, retaining the energy delivery element in a portion of the internal jugular vein contained within a carotid sheath, and energizing the energy delivery element to transvenously direct energy to target contents of the carotid sheath external to the internal jugular vein. The energy may be directed to a carotid artery within the carotid sinus sheath, and/or to a carotid sinus nerve or nerve branch within the carotid sinus sheath, to nerve branches emanating from carotid artery baroreceptors, and/or to a vagus nerve or associated nerve branch within the carotid sinus sheath. In some of the disclosed embodiments, a bi-lateral system is employed, in which a second electrode or other second energy delivery element is introduced into a second internal jugular vein and retained in a portion of the second internal jugular vein contained within a second carotid sheath. The second energy delivery element is energized to direct energy to contents of the second carotid sheath external to the second internal jugular vein.

The '495 application additionally describes the use of shielding to minimize collateral stimulation of unintended targets. In one embodiment, a shield is positioned at least partially surrounding the carotid sinus sheath. The shield blocks conduction of energy beyond the sheath during energization of the energy delivery element. In another embodiment, an insulative material is delivered into extravascular space adjacent to the internal jugular vein. The insulative material defines a channel within the extravascular space. Energizing the energy delivery implant causes energy to conduct along the channel to the target contents of the sheath.

The '495 application further discloses that the system may include a plurality of electrodes disposed on the lead, the electrodes including a first array and a second array, wherein the first and second arrays are positioned such that when the first array is positioned in the internal jugular vein to direct stimulation energy transvascularly to a vagus nerve in the carotid sheath, the second array is positioned to direct stimulation energy transvascularly towards a carotid artery, to stimulate, for example, the baroreceptors, baroreceptor afferents, carotid sinus nerve and/or associated nerve branch within the carotid sheath. In other embodiments, the same array of electrodes delivers stimulus to each of the target structures within the carotid sheath.

In the present application, embodiments are described which use an intravascular lead to deliver energy for stimulating nervous system targets using energy delivery elements (e.g. electrodes) that are in direct contact with the nervous system targets. These embodiments preferably position the lead within the internal jugular vein. Several of the disclosed embodiments utilize the relatively fixed geometric relationship between the carotid artery/carotid sinus bulb and the internal jugular vein within the carotid sheath by positioning the lead within a portion of the internal jugular vein disposed within the carotid sheath. The nervous system targets may include the carotid artery/carotid sinus bulb and/or associated baroreceptor afferents, and/or surrounding nervous system targets in the region of the internal jugular vein, such as the carotid sinus nerve and/or associated nerve branches and/or the vagus nerve and/or associated nerve branches.

Various arrangements are described for enabling passage of stimulation energy from a lead disposed in the internal jugular vein to the nervous system targets. In some of the disclosed embodiments, stimulation energy travels along a conductive bridge that extends from the internal jugular vein (IJ) to the nervous system target. In many of these embodiments, the conductive bridge is formed by extending a portion of the IJ lead through a sidewall of the internal jugular vein. Electrodes positioned on an electrode carrying element are electrically coupled to the lead and are placed in contact with the nervous system target such as the carotid artery or the vagus nerve. The lead and electrode(s) conduct energy from the pulse generator to the nervous system target.

In other embodiments, a conductive bridge is formed using a conductive material extending between the internal jugular vein and the carotid artery, so that current passing from electrodes in the internal jugular vein will conduct through the wall of the internal jugular vein, and across the conductive bridge to the carotid artery.

In other disclosed embodiments, energy is relayed from the IJ lead to another device disposed within or surrounding the target structure. The relayed energy may be converted to a form suitable for use in stimulating the target structure. In one exemplary embodiment, energy is relayed from a first component in the internal jugular vein to a second component positioned in or surrounding the common carotid artery for use in stimulating nervous system targets in the region of the carotid bulb.

### BRIEF DESCRIPTION OF THE DRAWINGS

FIG. 1A schematically illustrates intravascular positioning of portions of the disclosed system for stimulation of the carotid artery or other nervous system targets in the region of the sinus bulb.

FIG. 1B schematically illustrates an internal jugular vein and carotid artery from within the region defined as 1B-1B in FIG. 1A, and shows intravascular positioning of an electrode for stimulation of the carotid sinus bulb.

FIG. 1C is a schematic cross-sectional top view of the vessels shown in FIG. 1B and further shows the electrode positioned in the carotid artery with its lead extending into the internal jugular via the walls of the carotid artery and internal jugular.

FIG. 2A is a side elevation view of the electrode of FIGS. 1A and 1B.

FIG. 2B is a perspective view showing the electrode of FIG. 2A and an electrode pusher within a deployment catheter. The catheter is shown as transparent.

FIG. 3 is a side elevation view showing a second embodiment of an electrode.

FIGS. 4A through 4E are a sequence of schematic drawings illustrating implantation of the electrode of FIG. 3.

FIG. 5 is a perspective view of a third embodiment of an electrode.

FIGS. 6A and 6B are a sequence of schematic drawings illustrating implantation of the electrode of FIG. 5.

FIG. 7A is a side elevation view of a fourth embodiment of an electrode positioned within a deployment catheter. The drawing also shows, in the lower portion of the drawing, the position of the electrode following its advancement through the vein and artery walls.

FIG. 7B is a perspective view of the electrode and deployment catheter of FIG. 7A.

FIG. 7C is a cross-section view of an artery showing the electrode following implantation.

FIG. 8A is a plan view of a fifth embodiment of an electrode carrying element.

FIG. 8B is a side view of a strut of the embodiment of FIG. 8A.

FIG. 9A is a perspective viewing showing the electrode carrying element of FIG. 8 positioned within a deployment catheter being advanced from a vein towards a nearby artery.

FIG. 9B is a cross-section view of a vein and artery, showing the electrode carrying element of FIG. 8 following deployment.

FIG. 9C is a perspective view of an exterior of artery showing the electrode carrying element in position. The lead is not shown in FIG. 9C.

FIG. 10A is a schematic side elevation view of an internal jugular vein and a carotid artery, illustrating an alternative embodiment of an electrode carrying element.

FIG. 10B is similar to FIG. 10A and shows a modified electrode carrying element.

FIG. 10C is similar to FIG. 10B, and shows an additional electrode carrying element on the vagus nerve.

FIG. 11 is a cross-section view of a vein and an artery showing a sixth embodiment of an electrode.

FIG. 12 shows a vein and an artery in transparent view, and shows a seventh embodiment of an electrode.

FIGS. 13A and 13B are side views of magnetically dockable implantation catheters.

FIG. 14 shows side and end views of an alternative magnetically dockable implantation catheter.

FIG. 15 schematically shows a side view of an internal jugular vein and carotid artery and illustrates formation of a conductive bridge within the extravascular space within the carotid sheath.

FIG. 16 is a posterior view of the region designated 1B-1B in FIG. 1A, illustrating positioning of alternative stimulation components in the vasculature.

FIG. 17A is a schematic view of an internal jugular vein and a common carotid artery illustrating one configuration of the stimulation components utilizing inductive coupling.

FIG. 17B is a schematic view of an internal jugular vein and a common carotid artery illustrating an alternative to the FIG. 17A embodiment.

FIG. 18 is a schematic view of an internal jugular vein and a common carotid artery illustrating one configuration of the stimulation components using light as an energy transfer mechanism.

FIG. 19 is a schematic view of an internal jugular vein and a common carotid artery illustrating one configuration of the stimulation components using acoustic energy as an energy transfer mechanism.

FIG. 20A is a perspective view of an embodiment of a stent type device employing microactuators for generating a baro response. FIG. 20B is an end view and a side elevation view of the stent of FIG. 20A.

### DETAILED DESCRIPTION

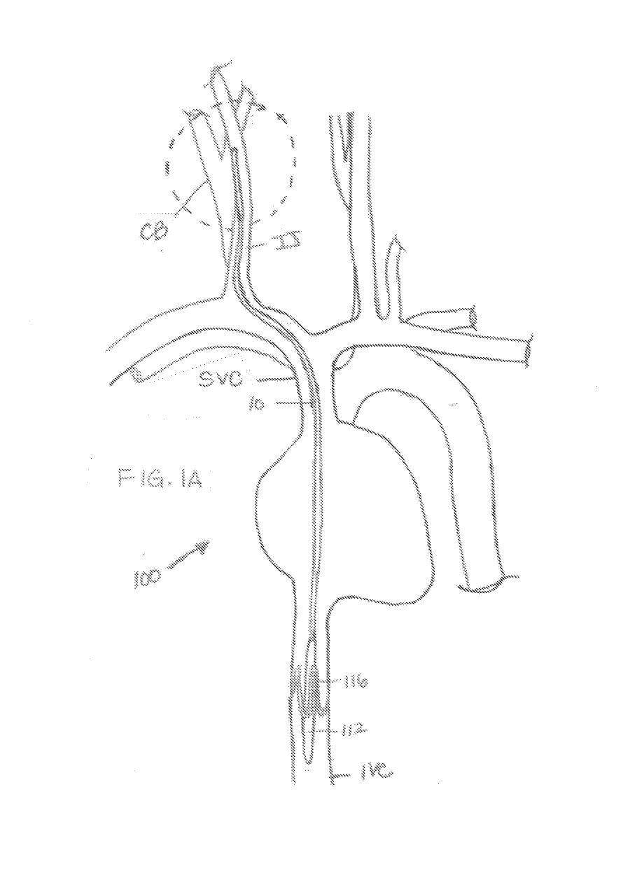

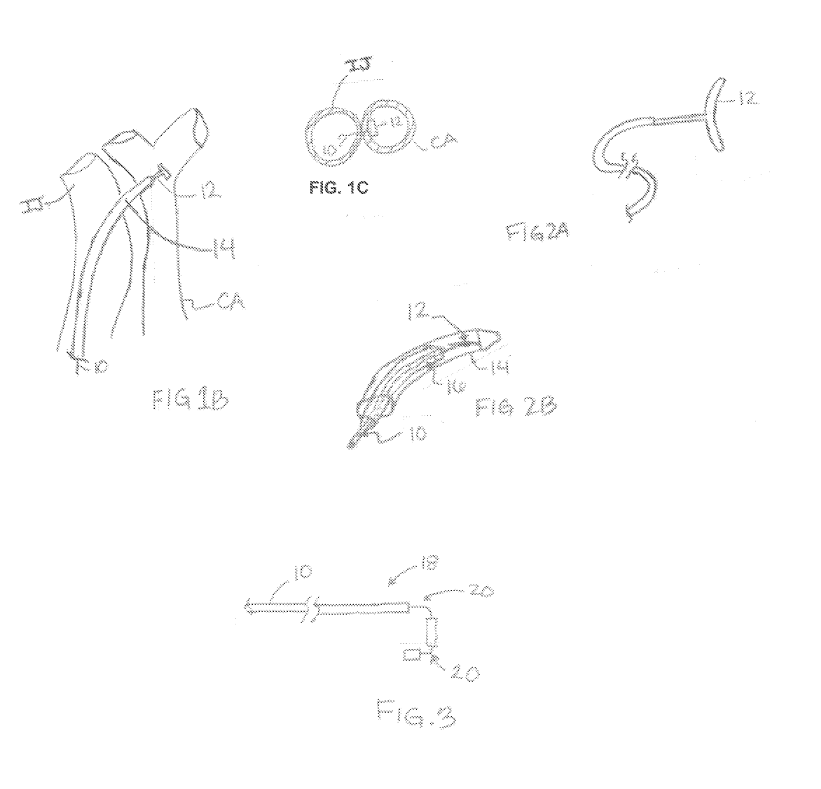

Referring to FIG. 1A, the disclosed designs for electrodes, leads, and other energy delivery elements may be part of a system 100 which includes a housing 112 containing the necessary pulse generator and associated electronics, circuitry, battery and related components and at least one lead 10 carrying some or all of the electrodes or other energy delivery elements needed to deliver electrical energy to target nervous system structures. In the illustrated embodiment, the housing 112 is positioned in the inferior vena cava (“IVC”), but it may alternatively be positioned in other vessels including, but not limited to, the superior vena cava (“SVC”), or the left or right subclavian vein (“LSV” or “RSV”). An anchor 116 is used to retain the housing within the vasculature. Features suitable for use with the system, including embodiments of leads, electrodes, housings and anchors are shown and described in the following patents and applications, each of which is incorporated herein by reference: U.S. Pat. No. 7,082,336 entitled IMPLANTABLE INTRAVASCULAR DEVICE FOR DEFIBRILLATION AND/OR PACING, U.S. 2005-0043765 entitled INTRAVASCULAR ELECTROPHYSIOLOGICAL SYSTEM AND METHODS, U.S. US 2005-0228471, entitled METHOD AND APPARATUS FOR RETAINING MEDICAL IMPLANTS WITHIN BODY VESSELS, U.S. Pat. No. 7,363,082, entitled FLEXIBLE HERMETIC ENCLOSURE FOR IMPLANTABLE MEDICAL DEVICES, U.S. US 2005-0154437, entitled IMPLANTABLE MEDICAL DEVICE HAVING PRE-IMPLANT EXOSKELETON, and U.S. 2007/0255379, entitled INTRAVASCULAR DEVICE FOR NEUROMODULATION.

The lead 10 extends from the housing 112 and is disposed within a blood vessel, preferably on the venous side, with its electrodes positioned to stimulate nervous system structures outside the vessel within which the lead is placed. In the embodiments described with reference to FIGS. 1B through 15, the lead is disposed within a vein, with the electrodes (or other energy delivery elements) positioned in contact with the nervous system target, such as an artery or a nerve. For example, the electrodes may be positioned in contact with the walls of the carotid artery, preferably at the carotid bulb CB, to allow electrical energy from the electrodes to stimulate the baroreceptors of the carotid artery, baroreceptor afferents associated with the carotid artery, and/or the carotid sinus nerve or associated nerve branches. In preferred embodiments, the electrode lead is delivered via the internal jugular IJ vein to the location in the neck at the common carotid bifurcation. The distal portion of the lead is extended through the wall of the internal jugular vein and positioned in contact with the carotid artery. Stimulation of the carotid artery, carotid baroreceptor afferents, carotid sinus nerves and/or associated nerve branches, or other associated nervous system structures or targets can activate a baro-response for the treatment of hypertension. The present application discloses leads having electrode carrying elements at their distal ends. The electrode carrying elements are designed to position and retain electrodes in contact with the target structure (e.g. an artery) while minimizing stimulation of unintended structures during therapy, and while also minimizing power consumption.

In a first embodiment shown in FIG. 1B, the lead 10 includes an electrode/electrode carrying element 12 in the form of a “t-bar” shaped element. According to this embodiment, a small-diameter catheter 14 is advanced through the internal jugular and pushed through the wall of the internal jugular (IJ) and into the arterial wall (CA). Once the distal end of the catheter 14 is inside the artery, a pusher 16 (FIG. 2B) is advanced to deploy the electrode 12 from the catheter 14. Lead 10 is withdrawn slightly to draw the electrode 12 into contact with the interior wall of the artery as shown in FIG. 1C. The top of the “T” forming the electrode may be slightly curved as shown in FIG. 2A to more closely conform to the arterial wall. Lead 10 extends through the internal jugular vein and is coupled to the pulse generator 112 (FIG. 1A). Additional electrodes may be implanted in similar fashion.

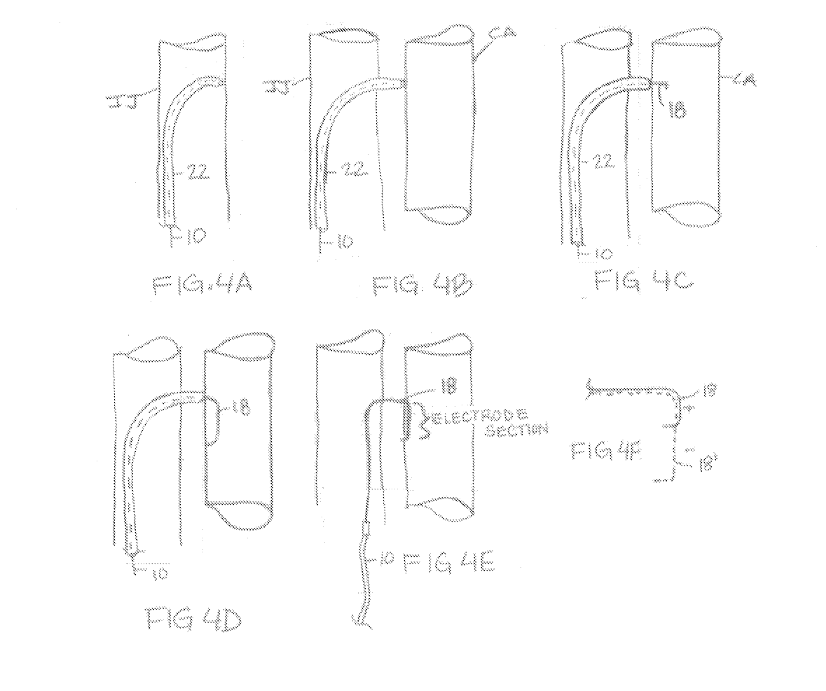

FIG. 3 illustrates an alternative electrode carrying element design 18 comprising a lead 10 with a distal end formed of an insulated nitinol wire. The wire is shape-set to have an approximate J-shape as shown. Insulation covering the wire is removed to form one or more spaced apart electrode regions 20. To implant the electrode carrying element 18, the J-shaped electrode is straightened and inserted into a delivery catheter 22. The catheter may be a tube of nitinol, PEEK, or other material, and it may have a distal end that is deflectable or pre-curved to allow it to be oriented for passage through the vessel wall. The catheter is then advanced to the internal jugular vein as shown in FIG. 4A, and then passed through the walls of the internal jugular and the carotid artery as shown in FIGS. 4B and 4C. The element 18 is advanced from the catheter into the carotid artery, assuming its shape-set form as it deploys. The J-shape of the element allows the electrode regions 20 (FIG. 3) to seat against the vessel wall, with the tip of the J engaging the interior surface of the vessel as shown. As shown in FIG. 4F, multiple such wires 18, 18′ may be simultaneously deployed through the catheter to position multiple electrodes against the carotid artery. The electrode carrying elements of the wires have varying lengths as shown, to maintain physical separation between electrodes of opposite polarity when the electrodes are implanted.

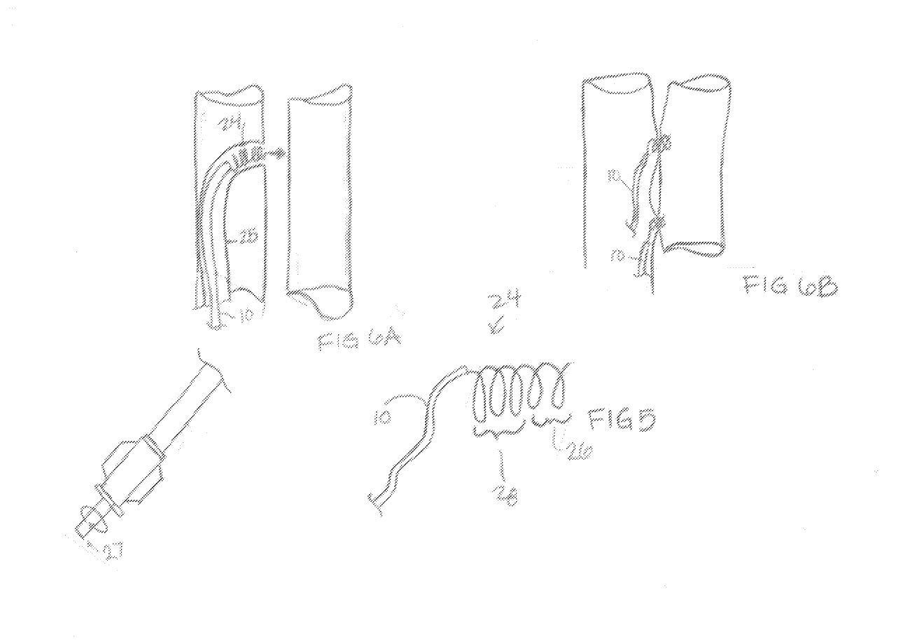

In an alternate embodiment shown in FIG. 5, lead 10 includes an electrode/electrode carrying element 24 having a corkscrew shape. A distal portion 26 of the corkscrew is conductive, whereas the proximal portion 28 of the corkscrew is insulated to minimize current delivery to unintended areas. To deploy the lead 10 and electrode 24, a delivery catheter 25 is advanced into the internal jugular vein and positioned with its distal opening in proximity to the vessel wall as shown in FIG. 6A (which shows the catheter as transparent to allow the lead and electrode to be seen). The lead 10 is advanced such that the distal tip engages the vessel wall. The lead 10 is rotated using a torquing instrument to screw the electrode through the wall of the internal jugular. Once the electrode exits the internal jugular, it is further advanced until it contacts the wall of the carotid artery, and it is again torqued until its distal portion 26 screws into the tissue of the carotid artery as shown in FIG. 6B. Additional electrodes are deployed in a similar way if needed, allowing for a bi-polar or tri-polar electrode arrangement

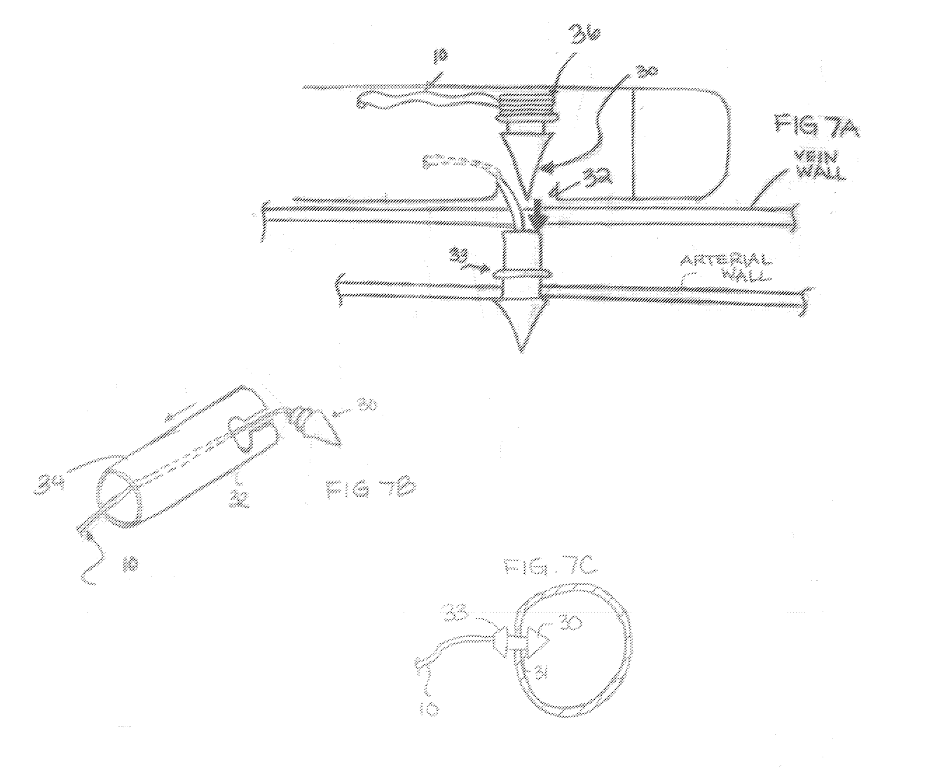

In yet another embodiment shown in FIG. 7A, electrode/electrode carrying element 30 comprises a pointed tip that may be laterally ejected through a sidewall opening 32 in the delivery catheter 34 (see arrow A) using a energy released from a compressed spring 36, or using driving force from a hydraulic or pneumatic source. Ejection of the electrode 30 drives it through the jugular vein and the carotid artery, leaving it embedded in the artery wall as shown in FIG. 7C. The shape of the sidewall opening 32 gives the distal portion of lead 10 a path through which to exit the catheter. After the electrode is ejected, the catheter is withdrawn from the body.

The electrode may include features to prevent the electrode from being inadvertently detached from its position in the arterial wall. For example, implantation of the electrode 30 may position a shoulder 31 and collar 33 on opposite sides of the arterial wall, to prevent the electrode from being withdrawn from or advanced further into the artery.

FIG. 8A illustrates an alternative electrode carrying element 40 which does not require penetration of the artery wall for implantation. Instead, the electrode(s) on the electrode carrying element at the distal end of the lead 10 is/are passed through the venous wall and positioned in contact with the exterior surface of the artery.

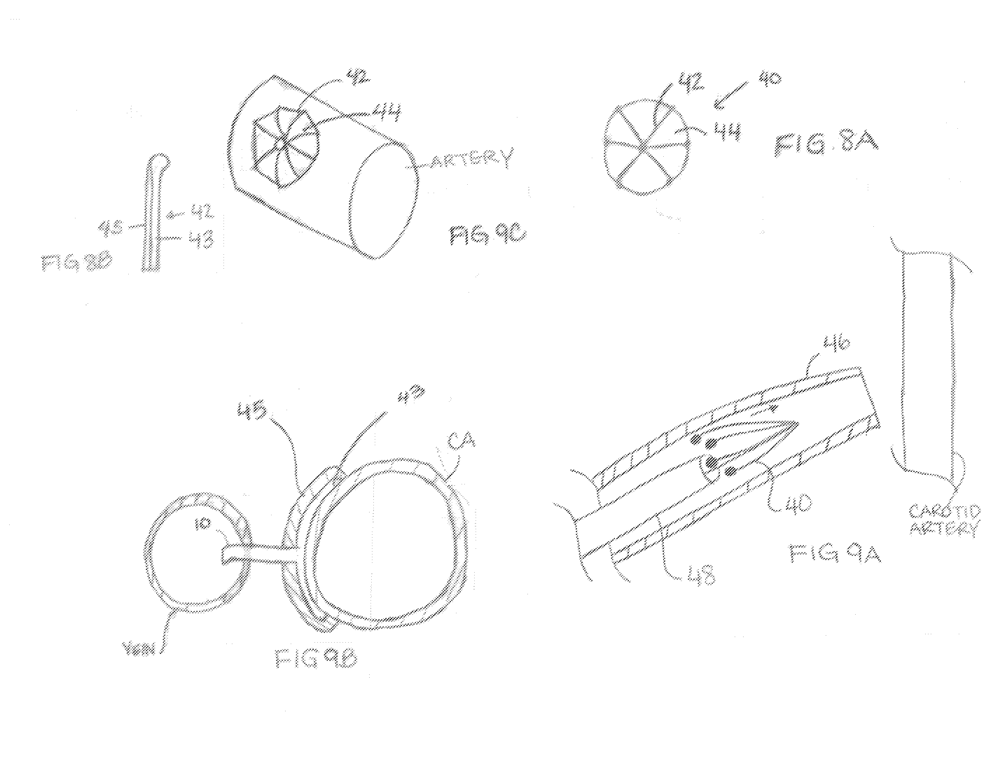

Electrode carrying element 40 is formed of conductive struts 42 (e.g. nitinol, stainless steel, platinum or other flexible conductive material) with non-conductive webbing 44 extending umbrella-like between the struts 42. Some or all of the struts have a conductive contact surface or electrode 43 and an insulating surface 45 (FIG. 8B). The struts have a preset shape that biases the electrode contact surfaces 43 against the exterior of the artery wall when the electrode carrying element is deployed, and that “pinch” the tissue so as to hold the electrode in place. Prior to deployment, the electrode carrying element 40 is collapsed and positioned in a deployment sheath 46, which is advanced through the wall of the internal jugular so that its distal end is positioned adjacent to, but external of, the carotid artery as in FIG. 9A. A pusher 48 is used to advance the electrode carrying element 40 out of the deployment sheath, causing the electrode 40 to expand. The expanded element 40 is advanced into contact with the outer surface of the carotid artery as shown in FIGS. 9B and 9C. In modified forms of this embodiment, the electrode carrying element may include additional engaging features, such as barbs on the struts, or a pin having a distal tip that penetrates the arterial and then expands to retain the electrode position. Adhesives may also be used to anchor the element to the exterior surface of the artery.

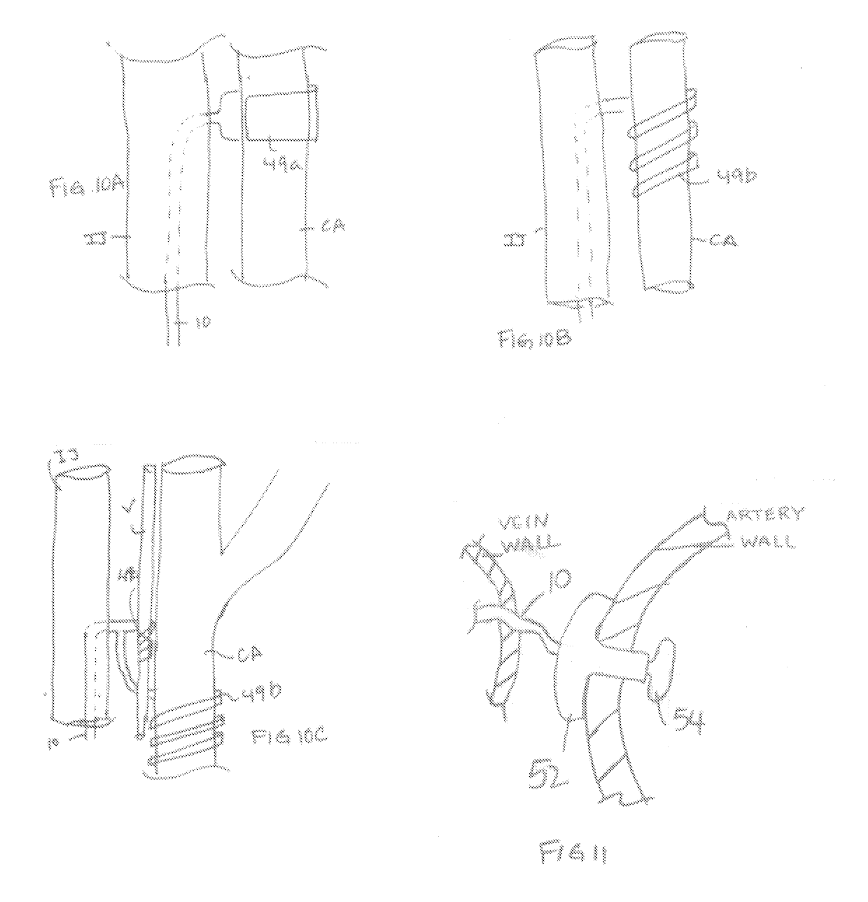

Other embodiments that do not require penetration of the carotid artery are shown in FIGS. 10A and 10B. As shown, the electrode carrying element comprises a ribbon-like cuff 40a having a free end shape-set so that is can curl partially or fully around the artery, or a helical cuff shape-set to coil multiple times around the artery. The electrode carrying elements may be passed through the wall of the internal jugular vein for deployment (e.g. in a straightened configuration contained within a sheath, in a step similar to that shown in FIG. 4B), or they may be introduced into the extravascular space through an incision through the skin (preferably in the neck). In the latter case, the electrode carrying element is connected to the distal end of the lead 10 after the lead has been delivered intravascularly to the internal jugular vein and after the lead tip has been passed through the wall of the internal jugular vein.

As shown in FIG. 10C, an electrode carrying element 49c on the lead 10 may be similarly coupled to another nervous system target such as the vagus nerve V. In this embodiment, the lead 10 branches into a first lead section 10a coupled to the carotid artery electrode carrying element 49b, and a second lead section 10b coupled to the vagus nerve electrode carrying element 49c. In other embodiments, separate leads passed through and extending from the internal jugular vein may be used for the vagus nerve and carotid artery. In still other embodiments, where carotid artery stimulation is not needed or is accomplished by other means, only a single lead is disposed in the internal jugular vein and is coupled to the electrode carrying element 49c.

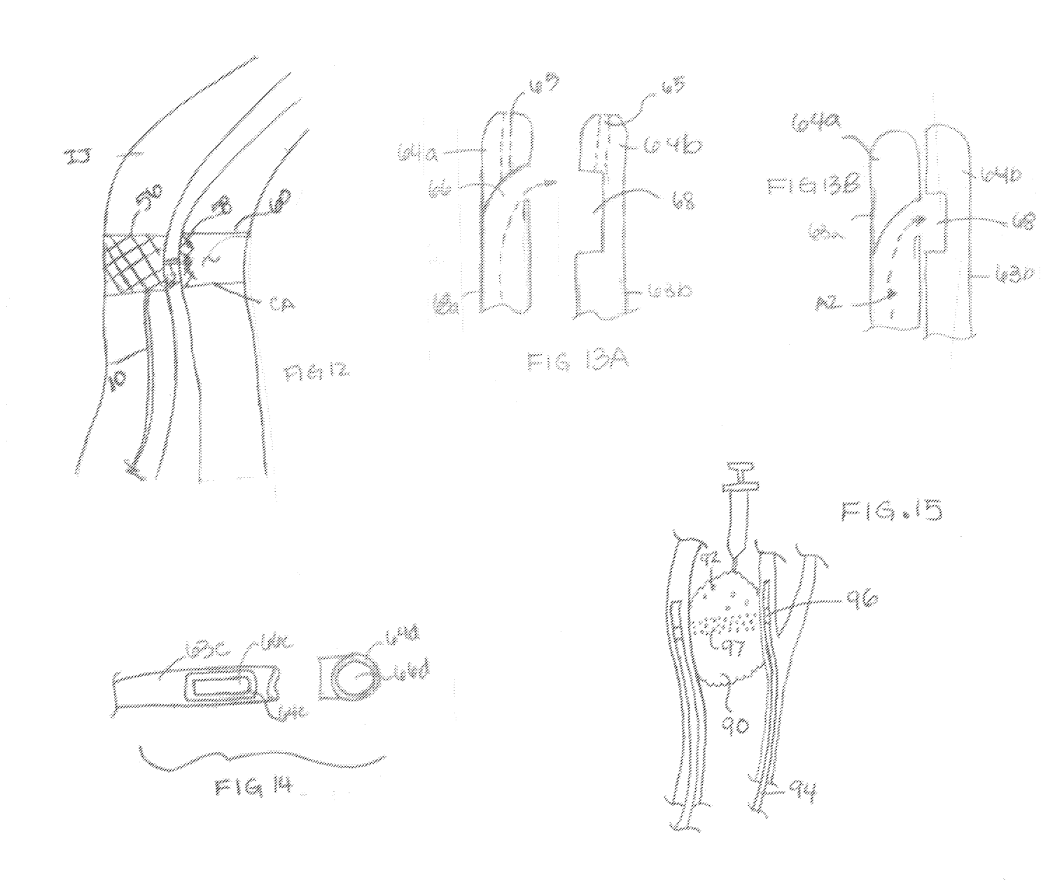

FIG. 11 shows an electrode embodiment which has a rivet-type configuration. According to this embodiment, the electrode includes a base portion 52 coupled to the electrode lead 10. The base portion 52 is introduced through the internal jugular vein and then inserted through the wall of the artery to the position shown. An insert 54 delivered through the artery is passed into an opening in the base portion 52 to lock the components together as shown. Alternatively, rather than an insert, the rivet may include an anchor that expands once inside the artery to retain the position of the rivet.

In an alternative embodiment shown in FIG. 12, lead 10 is anchored in the internal jugular vein using a stent like anchor 56. Electrodes 58 are mounted to a similar stent-like anchor 60 positioned in the carotid artery. A pin 62 extending from the anchor 56 is advanced through the walls of the internal jugular vein and the carotid artery and is placed in electrical contact with conductors on the anchor 60 to energize the electrodes 58.

Deployment of any of the disclosed electrodes can be facilitated by drawing the vein and artery into close proximity to one another, to allow passing of instruments, electrodes etc. from one vessel to the other. To help bring the vessels closer together, catheters 63a, 63b having magnetic-tips 64a, 64b of the type shown in FIG. 13A may be used during electrode positioning. During use, catheter 63a is introduced into the internal jugular vein and catheter 63b is introduced into the carotid artery. Each catheter may include a guide wire lumen 65 to allow its passage over a guidewire.

Once the magnetic tips 64a, 64b of the catheters magnetically engage or dock to one another (FIG. 13B), the electrode is pushed through a channel 66 in the catheter 63a, advanced through the walls of the vein and artery, and passed into a receiving window 68 in the catheter 63b or directly into the artery. Mechanically docking the catheters in this way positions the channel 66 and window 68 against the vessel walls, which are themselves drawn together, facilitating transfer of the electrode carrying element from one catheter to the other as illustrated by arrow A2.

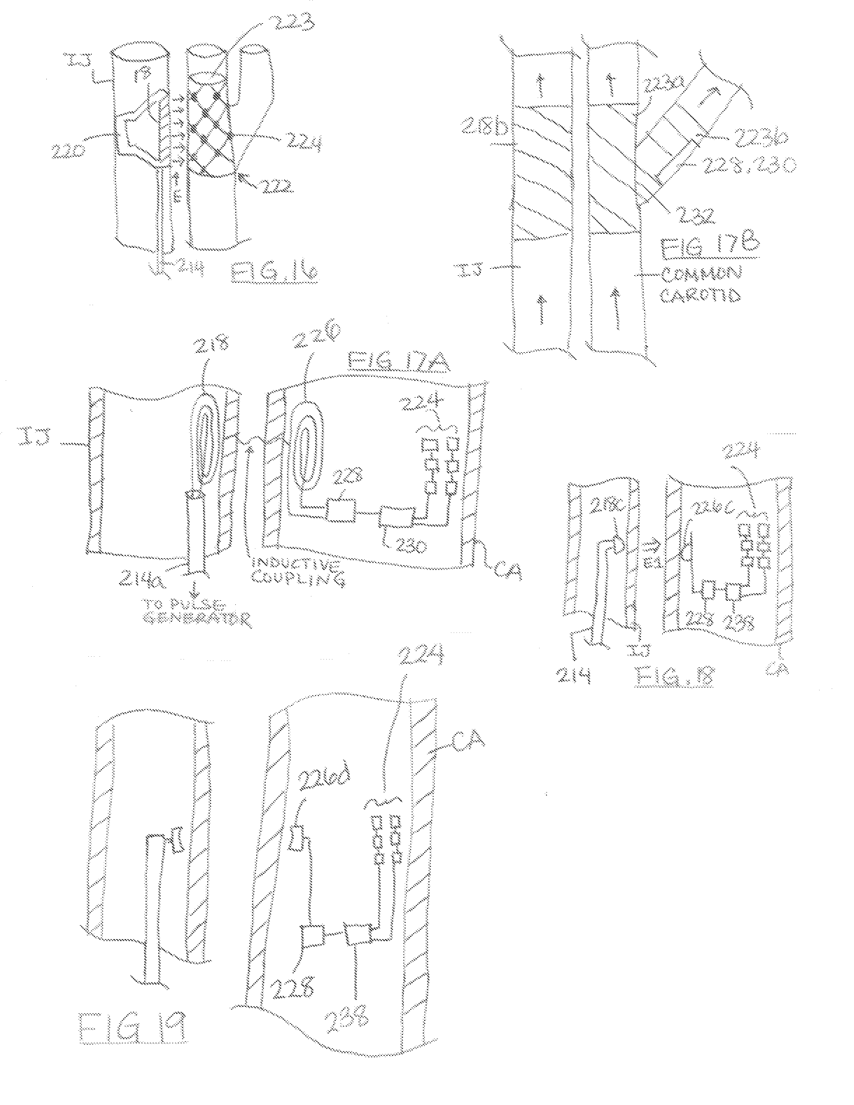

The catheters may be repositioned one or more times to allow implantation of additional electrodes. This embodiment can eliminate the need for passing a lead delivery catheter from the internal jugular vein into the carotid artery. In alternative docking catheter designs, a magnetic catheter 63c may be constructed as in FIG. 14, in which an annular (or other shaped) magnet 64c, 64d borders a window 66c, 66d in the catheter through which an electrode may be advanced or received.

Although the embodiments described thus far have been described as ones which deliver electrical energy, it should be understood that these embodiments may be adapted to deliver other forms of energy to the nervous system targets. For example, the energy delivery element coupled to the lead 10 may include one or more piezoelectric transducers that transmit ultrasonic or other acoustic or vibrational energy to the nervous system target when a potential is applied to them, so as to mechanically stimulate the nervous system target.

For example, in a modification to the FIG. 9A-9C embodiments, the contact surfaces 43 of the struts 42 may be piezoelectric elements, while the opposed surfaces 45 of the struts include electrodes electrically coupled to conductors in the lead 10. Alternatively, piezoelectric elements, such as those formed of piezoelectric film, may be positioned on the webbing 44 between the struts with associated electrodes positioned on the struts or on the webbing 44. Acoustic/ultrasonic/mechanical stimulation of the nervous system target can be achieved by applying a potential across the piezoelectric elements using the electrodes as is known in the art.

In other embodiments, an injected substance may be used to form the conductive bridge between the internal jugular vein and the nervous system targets. In such an embodiment, substance 90 is injected into the extravascular space between the internal jugular vein and the carotid artery. The injection site for deposit of the substance is preferably within the carotid sheath, since the sheath will facilitate containment of the substance. The substance 90 contains magnetic or paramagnetic particles 92 which are also electrically conductive. Catheters 94 having distally positioned magnets 96 are intravascularly delivered into the internal jugular vein and the carotid artery. Catheters having permanent magnets or energizable electromagnetic coils may be used for this purpose. The magnets preferably have opposite polarity from one another. The magnets 96 cause the magnetic particles 92 to orient themselves with the magnetic fields of the magnets, forming a conductive bridge 97 between the internal jugular and the carotid artery. Examples for the substance containing the particles include thixotropic materials (which have low viscosity when subjected to stresses during injection using a syringe, but which increase in viscosity once injected), and polymeric substances or gels that may be cured using light, energy, or other substances following injection (for example using a light or other energy transmitting element passed through the injection site or positioned in the internal jugular vein for use in curing the substance), so that the position of the conductive bridge becomes fixed.

Thus, electrical energy delivered by electrodes disposed within the IJ (as described in co-pending U.S. application Ser. No. 12/413,495, filed Mar. 27, 2009 and entitled SYSTEM AND METHOD FOR TRANS VASCULARLY STIMULATING CONTENTS OF THE CAROTID SHEATH) will preferentially conduct across the bridge_to the carotid artery. The position for the bridge may also be selected such that energy conducting across the bridge will also or alternatively stimulate the vagus nerve.

### DETAILED DESCRIPTION

FIGS. 16-20B show systems which system uses two separate components for delivering stimulation energy to the carotid baroreceptor region. Referring to FIG. 16, the first component is an intravascular lead 214 that is delivered through the internal jugular vein to about the location of the carotid bifurcation. This lead 214 is provided with an anchoring mechanism 220 allowing it to be optimally positioned and anchored. The lead includes an energy generating component 218 that is positioned such that it may effectively transfer energy E from the internal jugular (IJ) across the interstitial space to a second component 222 located in the carotid artery (CA). The energy generating component 218 can be composed of various classical energy transmission technologies, including inductive, RF, light, acoustic, magnetic, electrical and mechanical. The lead 214 structure also possesses insulation characteristics such that the energy transferred can be optimally targeted in a focused way with minimal attenuation due to stray leakage pathways.

The second component 222 is an energy receiving component positioned to receive the energy E from the first component 218 and relay that energy in some form towards target structures. In preferred embodiments, the second component 222 is positioned for stimulation of the baroreceptors of the carotid bulb, or the associated carotid sinus nerves, to activate a baro-response for the treatment of hypertension. The second component 222 is designed to be anchored within its intended blood vessel, and in a preferred embodiment is similar in structure to a carotid stent having radially expandable walls that anchor the second component within the carotid artery. It should be noted that while “stent-like” devices such as the component 222 resemble stents in the sense that they are expandable so as to radially engage a vascular wall, the anchors need not have the hoop strength possessed by conventional stents as needed by such stents to maintain patency of the diseased vessels within which they are conventionally implanted.

The stent 223 is delivered through the common carotid to the location of the carotid bifurcation and positioned such that the main surfaces of the stent are co-radial with the baroreceptor region of the carotid artery, allowing for full circumferential contact with the baroreceptor region. In preferred embodiments, the stent includes active electrodes 224 used to deliver energy to the target area in the baroreceptor region. This energy can be any of the classical energy forms, preferably electrical, but alternatively acoustic, light, magnetic or mechanical.

The stent 223 is provided with an energy receiving mechanism allowing it to receive directed energy from the lead in the internal jugular and utilize that energy in some form for simulation. An energy transfer/distribution and energy conversion mechanism allowing the transvascularly delivered energy to be effectively distributed to the stimulation electrodes or other energy delivery mechanisms. In the preferred embodiment, the energy is distributed to the surface of the stent in contact with the artery wall. The stent 223 has insulating characteristics configured such that the energy directed to the carotid artery wall does not have leakage pathways back into the blood pool.

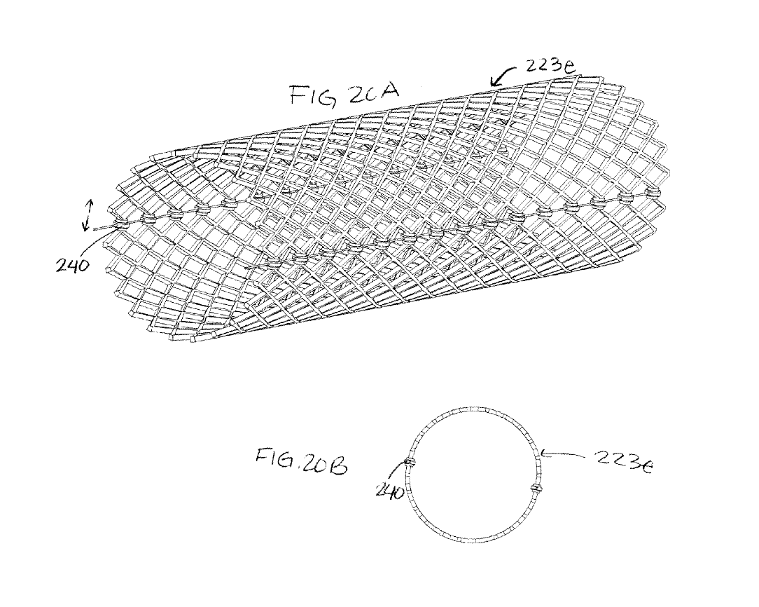

Referring to FIG. 17A, one embodiment of the lead includes an induction coil as the energy generating element 18a. The second component 222 includes a receiving coil 226. The lead 214a and second component 222 are positioned in the internal jugular (IJ) and common carotid (CC), respectively, for optimal energy transmission from the lead 214a to the second component 222. Energy E is generated by sending electrical energy through coil 218a at such a frequency to optimize energy transfer at the required stimulation frequency, duty cycle, and amplitude to the coil 226 within the constraints of the geometry of the internal jugular vein. The energy is transferred between the coils 218a, 226 by inductive coupling. The receiving coil 226 converts the energy back to electrical pulses. Energy receiving, collection and storage circuitry 228 and energy control and distribution circuitry 30 deliver these pulses to insulated electrodes 224 located around the outer circumference of the stent (not shown in FIG. 17A). Electrodes 224 preferably comprise both cathodes 224a and anodes 224b.

In an alternative shown in FIG. 17B, the induction coil 218b and receiving coil 226b are integrated into stent structures used to anchor them in the corresponding blood vessels. Here the carotid stent is designed as a bifurcated stent, which may be a single piece stent or which may be a modular stent allowing serial implantation of each stent piece. For example, a first piece 223a may be deployed in the common carotid, with its distalmost end extending into the internal carotid. First piece 223a includes an opening 232 positioned in alignment with the bifurcation to the external carotid. A second piece 223b is passed through the opening, deployed in the external carotid as shown, and electrically or electrical and mechanically coupled to the first piece 223a. The circuitry 228, 230 associated with the receiving coil 226b may be supported by the second piece 223b as shown, or elsewhere on the carotid side implant. The stents may be actively expandable or self-expandable into contact with the vessel wall.

FIG. 18 shows an alternative embodiment utilizing light as the energy transfer mechanism. For example, the energy generating component is a source of optical energy, such as an LED 218c selected for optimal transmission in the body and through the vessel tissue. The LED is positioned on the lead 214 in the internal jugular. Energy receiving component 226c comprises a photo detector on the stent or other carotid anchor. Signals generated by the photo detector in response to detection of optical energy E1 from the LED are converted by circuitry 228, 230 to stimulation energy for electrodes 224.

FIG. 19 shows an alternative embodiment utilizing ultrasonic energy as the energy transfer mechanism. Here, the energy generating component is an ultrasonic crystal 218d, selected for optimal transmission in the body and through tissue. The crystal 218d is positioned on the lead in the internal jugular. Energy receiving component 226d is an ultrasonic receiving crystal anchored in the carotid as discussed. Crystal 218d is energized to generate acoustic waves which are directed towards the crystal 226d. The resulting vibration of crystal 226d generates electrical pulses that are used to for stimulation energy for electrodes 224.

Although the disclosed embodiments use electrodes to deliver the desired stimulation energy, any of the disclosed embodiments may be modified to deliver mechanical energy to the carotid artery to activate a baroreceptor response For example, the energy received from the first component may be used to activate microactuators 240 on the stent 223e as shown in FIGS. 20A-20B. The microactuators may be elements that vibrate or undergo shape changes when exposed to an electrical potential. For example, the microactuators 240 may be piezoelectric elements, or nitinol elements formed to expand the stent when resistively heated. Alternatively, the actuators might comprise one or more small pumps that pump fluid from reservoirs into bladders on the stent, to impart pressure against the surrounding vessel well.

When activated, the microactuators 240 can vibrate or mechanically expand the stent, causing the carotid to experience mechanical pressure and to thereby activate a baroreceptor response and/or they can generate acoustic signals oriented towards target neurological structures.

While various embodiments of the present invention have been described above, it should be understood that they have been presented by way of example, and not limitation. It will be apparent to persons skilled in the relevant art that various changes in form and detail may be made therein without departing from the spirit and scope of the invention. This is especially true in light of technology and terms within the relevant art(s) that may be later developed. Thus, the present invention should not be limited by any of the above-described exemplary embodiments, but should be defined only in accordance with the following claims and their equivalents. The terms “first,” “second” and the like, where used herein, do not denote any order, quantity, or importance. In references to “first blood vessel”, “second blood vessel” etc., the first and second blood vessels may be different blood vessels or they may be the same blood vessel unless otherwise specified.

For example, the description of FIGS. 16 through 20B has largely discussed use of the system in the internal jugular and carotid artery, but has applications elsewhere in the vasculature. In other embodiments, the components are intravascularly positioned to stimulate any number of other neurological targets. Moreover, additional features may be added to the disclosed components to supplement stimulation capabilities, and the various embodiments can be combined in a number of ways to form additional embodiments. For example, in the FIGS. 16 through 20B embodiments, the first component may include electrodes positioned in the internal jugular for stimulation of carotid sinus nerve targets (e.g. the carotid sinus nerves or associated baroreceptors) and/or either the first or second component can include electrodes used to stimulate the vagus nerve. In other embodiment, an electrode on lead 10 may be used to stimulated the vagus nerve (as in FIG. 10C) and a device of the types shown in FIGS. 16 through 20B could be used to stimulate the carotid artery, carotid baroreceptor afferents, carotid sinus nerves etc. using energy relayed from the lead 10. As a further alternative, in the FIG. 16-20B embodiments, the second component may be placed around the carotid artery to deliver electrical, acoustic, ultrasonic, mechanical, or other forms of energy to the carotid artery in response to transmission of energy or signals from the first component in the internal jugular vein. In further embodiments, the system may be configured to stimulate the aortic baroreceptors, which are also responsible for providing signals to the brain for regulating systemic/peripheral blood pressure. Moreover, although the present application describes use of a lead in one of the internal jugular veins, the disclosed embodiments are equally suitable for use in bi-lateral systems (as described in the '495 application), in which leads extend into each of the internal jugular veins.

Any and all patents, patent applications and printed publications referred to above, including patent applications identified for purposes of priority, are incorporated herein by reference.

1. A method for intravascularly stimulating contents of the carotid sheath, comprising:
intravascularly advancing a lead into an first blood vessel;
forming an opening in the wall of the first blood vessel, and extending a portion of the lead through the opening;
positioning an energy delivery element into contact with a second blood vessel different from the first blood vessel;
coupling the lead to the energy delivery element; and
stimulating the second blood vessel using the energy delivery element.

2. The method of claim 1, wherein the first blood vessel is an internal jugular vein and the second blood vessel is a carotid artery.

3. The method of claim 1, wherein the energy delivery element is positioned in contact with a portion of the carotid artery disposed within the carotid sinus sheath.

4. The method of claim 1 wherein the energy delivery element is an electrode, and wherein stimulating the second blood vessel includes conducting energy to the second blood vessel using the electrode.

5. The method of claim 1, wherein the energy delivery element is a piezoelectric transducer, and wherein stimulating the second blood vessel includes delivering mechanical energy to the second blood vessel using the piezoelectric transducer.

6. The method of claim 1, wherein the energy delivery element is positioned on a distal portion of the lead, and wherein the method includes extending the energy delivery element through the wall of the first blood and into the second blood vessel.

7. The method of claim 6, further including anchoring the energy delivery element within the second blood vessel.

8. The method of claim 1, wherein the energy delivery element is positioned on a distal portion of the lead, and wherein the method includes extending the energy delivery element through the wall of the first blood and into contact with an exterior surface of the second blood vessel.

9. The method of claim 8, further including retaining the energy delivery element in contact with the exterior surface of the second blood vessel.

10. The method of claim 1, further including the step of extending a conductive bridge between the first and second blood vessels, and wherein the method includes conducting energy from an electrode disposed on the lead in the first vessel through the wall of the first blood vessel, and across the conductive bridge to the second blood vessel.

11. The method of claim 10, wherein extending the bridge includes injecting a conductive material into the extravascular space between the first and second blood vessels.

12. The method of claim 1, further including the step of delivering energy to a nerve disposed external to the first blood vessel.

13. The method of claim 12 wherein the nerve is the vagus nerve.

14. The method of claim 1, further including positioning a pulse generator in a third blood vessel, and electrically connecting the lead to the pulse generator.

15. An intravascular system for stimulating nervous system targets, comprising:
a pulse generator positionable within a blood vessel;
a lead coupled to the pulse generator and proportion to extend to a second blood vessel different from the first blood vessel;
an energy delivery element coupled a distal portion of the lead, the lead being extending through the wall of the second blood vessel to position the energy delivery element positionable in contact with a wall of a third blood vessel.

16. The intravascular system of claim 15, wherein the energy delivery element is engageable with an exterior surface of the second blood vessel.

17. The intravascular system of claim 15, wherein the energy delivery element positionable within the interior of the second blood vessel.
